(19) United States
(12) Patent Application Publication
(10) Pub. No.: US 2025/0235342 A1
(43) Pub. Date: Jul. 24, 2025

(54) HAND ORTHOSIS

(71) Applicants: TP-ORTHO GMBH, Bad Neuenahr-Ahrweiler (DE); MASALO KG, Lueneburg (DE)

(72) Inventor: Thomas PUETZ, Bad Breisig (DE)

(21) Appl. No.: 19/034,459

(22) Filed: Jan. 22, 2025

(30) Foreign Application Priority Data
Jan. 24, 2024 (EP) 24153635.8

(51) Int. Cl. A61F 5/01

(52) U.S. Cl. CPC A61F 5/0118; A61F 2005/0167

(57) ABSTRACT

The invention relates to a hand orthosis (10), with at least one finger receiving element (12), which is designed to receive, in particular enclose, at least one finger (102) of a hand (100) at least in certain areas.

The invention relates to a hand orthosis according to the preamble of claim 1, an orthosis assembly according to the general term of claim 12 and a method for adjusting according to the general term of claim 14.

Orthoses are medical aids known from the medical field that are used as externally applied devices to influence the structural and/or functional properties of the neuromuscular and/or skeletal system, for example to support therapy and/or healing after an injury and/or an operation on a part of the body, such as a hand. For example, hand orthoses are used in the therapy of carpal tunnel syndrome in order to increase the chances of success of the therapy and to restore and/or maintain the functionality of the wrist as quickly as possible and/or to strengthen the muscles of the hand and/or forearm during the therapy.

The hand orthoses known from the prior art are designed to immobilize the area of the hand and/or the forearm, in particular the fingers and/or the wrist, completely or at least partially during therapy. However, complete or at least partial immobilization of the hand can lead to the hand being immobilized in a position that is unfavorable for therapy and/or in an unnatural position, which can prolong the duration of therapy and/or reduce the success of therapy and/or even result in additional negative health consequences, for example an exacerbation of the symptoms of carpal tunnel syndrome. In particular, immobilization of the hand can lead to weakening of the hand and/or forearm muscles.

Furthermore, the hand orthoses known from the prior art are not suitable for treating a so-called drop hand, which can be the result of trauma or a stroke, for example, or for preventing a drop hand during the therapeutic treatment of other diseases or injuries in the hand area. The phenomenon of the drop hand describes a hand position in which the wrist and/or one or more fingers can no longer be sufficiently extended. This long-term angled position of one or more fingers and/or the wrist, known as a drop hand, can, as already described, reduce the success of therapy, prolong the duration of therapy and/or even lead to a worsening of the symptoms, in the worst case even to nerve damage.

The task underlying the invention is thus to provide an improved hand orthosis for increased therapeutic success and/or a shortened duration of therapy.

The problem is solved by a hand orthosis of the type mentioned at the beginning, wherein the hand orthosis comprises an adjusting device which is set up to generate a corrective force acting on the finger receiving element, by means of which the at least one finger can be brought into a corrected position.

Because the hand orthosis comprises an adjusting device, the position of a hand on which the hand orthosis according to the invention is applied can be corrected by means of a corrective force generated by the adjusting device, the corrective force and the movement of the hand into the corrected position preventing, in particular, the hand from being immobilized by complete immobilization in a position and/or posture that negatively influences the duration and/or success of the therapy. By generating a corrective force, the hand can also be brought into a position that prevents the occurrence of a drop hand by stretching the fingers and/or the wrist of the hand through a countertraction and a resulting controlled movement of the wrist and/or one or more fingers, so that, for example, irritation and/or strain in the area of the carpal tunnel is reduced and/or avoided and/or degradation of the forearm muscles is reduced and/or avoided, so that the success of therapy, for example after an operation and/or after an injury, can be maximized and the duration of therapy can be minimized and healing is thus optimally supported. The adjusting device of the hand orthosis and the controlled movement of one or more fingers or the entire hand that can be generated as a result can thus be used to dynamize the hand position and/or finger position instead of completely or partially immobilizing the hand.

Preferably, the adjusting device of the hand orthosis is designed to bring the hand and/or the wrist into a corrected position by means of the generated corrective force as an alternative or in addition to the at least one finger. The at least one finger receiving element of the hand orthosis is preferably designed as a finger ring, in particular as an elastic finger ring. The at least one finger holder can, for example, be designed as a loop, in particular as a ring-shaped loop, which encloses the finger completely or at least partially along its circumference and/or lies closely against the respective finger over its entire surface. Preferably, the finger receiving element is designed to transfer the corrective force generated by the adjusting device to the respective finger. In particular, the finger receiving element is designed to exert a tensile and/or compressive force on the respective finger as a result of the corrective force acting on the finger receiving element and in this way to push and/or pull the respective finger into a corrected position. Preferably, the corrective force generates a corrective movement of the respective finger into the corrected position. Alternatively or additionally, the corrective force can lead to a corrective movement of the entire hand, in particular the wrist, in order to bring the hand and/or the wrist into a corrected position.

Preferably, the hand orthosis comprises several finger receiving elements, in particular two, three, four or five finger receiving elements, which are each assigned to one finger and/or each enclose one finger, in order to be able to exert the corrective force generated by means of the adjusting device on several fingers simultaneously, so that several fingers and/or the entire hand and/or the wrist can be brought into an intended corrected position. In this way, it is possible to move and/or stabilize and/or immobilize and/or bring at least one finger and/or at least one finger joint and/or at least one wrist and/or at least one hand of a patient into an intended corrected position by means of the hand orthosis in such a way that a therapy of the respective hand carried out by means of the hand orthosis has a higher probability of success and/or the duration of therapy can be shortened, in particular by preventing a drop hand. Preferably, the hand and/or the wrist and/or the finger is brought into a neutral position and/or held in a neutral position by means of the corrective force, so that a drop hand can be avoided. Preferably, the hand orthosis, in particular by generating the corrective force by means of the adjusting device, relieves the hand and/or the finger and/or the wrist.

The hand orthosis can be made entirely of one material, for example an elastic or a non-elastic material. In addition, the hand orthosis can be made of different materials, in particular a mix of materials, whereby the hand orthosis, for example different areas of the hand orthosis, can be composed of elastic and non-elastic materials. Elastic materials are characterized by the fact that they undergo reversible stretching when force is applied. Non-elastic materials are characterized by the fact that due to their non-elasticity, in particular due to their higher strength and/or higher tensile and/or compressive force stability, they have no elastic deformability and do not stretch or only stretch slightly when force is applied. The at least one finger receiving element of the hand orthosis is preferably made of an elastic material and/or comprises an elastic material at least in some areas. For example, the finger receiving element can be made of silicone, neoprene, elastomer and/or rubber.

In a preferred embodiment of the hand orthosis according to the invention, the hand orthosis comprises at least one connecting element which connects the adjusting device to the at least one finger receiving element and is designed to transmit the corrective force generated by means of the adjusting device to the finger receiving element. Preferably, the connecting element extends along the back of the hand and/or contacts the back of the hand at least in certain areas. In particular, the connecting element lies flat and close to the back of the hand. In addition, the connecting element can extend at least in sections along one or more fingers of the hand and/or contact one or more fingers at least in certain areas and/or lie closely against one or more fingers at least in certain areas.

Preferably, the connecting element is connected to the adjusting device at a first end and/or is attached to the adjusting device. The fact that one end of the connecting element is connected to the adjusting device and/or is attached to the adjusting device means that the corrective force generated by the adjusting device can be transmitted to the connecting element, in particular a tensile force can be transmitted to the connecting element. Preferably, the connecting element has a second end which is connected to the at least one finger receiving element and/or is attached to the at least one finger receiving element. If the hand orthosis has several finger receiving elements, the connecting element preferably has a number of ends corresponding to the number of finger receiving elements, so that the connecting element is connected to each of the several finger receiving elements and/or attached to each of the other finger receiving elements. As a result of the connecting element being connected to the one or more finger receiving elements and/or attached to the one or more finger receiving elements, the corrective force generated by means of the adjusting device, which is transmitted to the connecting element in particular via the first end of the connecting element, can be transmitted to the one or more finger receiving elements. In particular, the connecting element and the ends of the connecting element connected to the one or more finger receiving elements transmit a tensile force to the finger receiving elements, by means of which the one or more fingers can be brought into an intended corrected position.

The connecting element can have a strip-like design, at least in sections. In particular, the connecting element between its end connected to the adjusting device and the one or more ends connected to the one or more finger receiving elements can have a width that changes over the length of the connecting element. In addition, the connecting element may comprise slot-shaped recesses which divide the connecting element into sections corresponding to the one or more fingers of the hand, in particular band-shaped sections. Preferably, the sections of the connecting element run along and/or parallel to the longitudinal extension of the respective finger.

In another preferred embodiment of the hand orthosis according to the invention, the connecting element is formed from an elastic and/or flexible material and/or comprises an elastic and/or flexible material. Alternatively or additionally, the connecting element is formed from a non-elastic and/or non-flexible material and/or comprises a non-elastic and/or non-flexible material. Preferably, the connecting element and the at least one finger receiving element are part of a common integral body. Preferably, the hand orthosis comprises at least one elastic connecting element which is, for example, in the form of a strap or band and connects the at least one finger receiving element to the adjusting device in such a way that a force generated by the adjusting device, in particular a tensile force, is transmitted to the at least one finger receiving element by pulling on the elastic connecting element with elastic deformation, in particular with stretching of the connecting element.

The connecting element is preferably made of one or more elastic materials, at least in certain areas and/or sections. In particular, the connecting element can be made of silicone, neoprene, an elastomer and/or rubber and/or comprise silicone, neoprene, an elastomer and/or rubber at least in some areas and/or sections. If the connecting element is made of an elastic material and/or the connecting element comprises an elastic material, the connecting element is elastically deformable and/or elastically stretchable under the action of force, in particular under the action of the corrective force, whereby the tensile stress in the connecting element caused by the elastic deformability due to the tensile force transmits the corrective force from the adjusting device to the at least one finger receiving element. Due to the elastic deformability and/or extensibility of the connecting element, the connecting element can in particular generate a spring effect.

The connecting element can alternatively or additionally be made of a material and/or comprise a material, at least in certain areas and/or sections, which has a lower elasticity than the elastic material described above. In particular, the connecting element can be made of and/or comprise a non-stretchable and/or non-elastic and/or solid, in particular tension-resistant and/or dimensionally stable, material. A non-stretchable and/or non-elastic material and/or a less elastic and/or less stretchable material exhibits no or at least reduced elastic deformability under the action of an external force, in particular under the action of the corrective force, and is therefore more dimensionally stable.

The connecting element and the at least one finger receiving element can be an integral part of a one-piece body. In particular, the connecting element and the at least one finger receiving element can together form a hand receiving element which contacts the hand at least in certain areas and/or encloses the hand at least in certain areas. A hand receiving element is preferably designed to hold the hand of a patient at least in certain areas; in particular, the hand receiving element can cover and/or contact a part of the hand, in particular the back of the hand, at least in certain areas, in particular by making surface contact.

Preferably, the connecting element is made of the same material and/or material mix as the at least one finger receiving element. Alternatively, the connecting element and the at least one finger receiving element can be two separate components of the hand orthosis that are connected to each other and/or attached to each other, which can also be made of different materials and/or material mixes.

Thus, the characteristics of the corrective force transmission from the adjusting device to the at least one finger can be individually changed by the choice of material and/or the combination of materials of the connecting element and/or the at least one finger receiving element, in particular by the choice of elasticity of the respective material and/or the arrangement in which area a respective material is used.

In a further preferred embodiment of the hand orthosis according to the invention, the adjusting device is designed as a mechanical adjusting device, wherein the corrective force can preferably be generated and/or adjusted by means of an adjusting member of the adjusting device and/or the adjusting member of the adjusting device can be fixed in a desired position for fixing an intended corrective force. Preferably, the corrective force can be adjusted individually and/or continuously by means of the adjusting member. The adjusting device can be designed, for example, as a ratchet lock and/or as a quick-release system and/or as a twist lock and/or as a rotary ratchet lock. In particular, the adjusting member can be designed as a BOA fastener known from shoes, for example

The adjusting device can be designed as a cable pull and/or comprise a cable pull. The adjusting device of the hand orthosis allows the corrective force acting on the at least one finger and/or on the hand to be quickly, precisely and easily individually adjusted, so that a desired corrective force can be set for the respective individual therapy for a patient. Preferably, the corrective force set by means of the adjusting device and/or the resulting corrected position can be locked and/or fixed in the desired position and/or position, so that a desired corrective force and/or a resulting desired corrected position is maintained by the hand orthosis in the long term. Preferably, the adjusting member of the adjusting device is lockable and unlockable for releasing and/or locking the desired corrective force and/or the desired corrected position, in particular by means of an unlocking movement and/or locking movement of the adjusting member.

Preferably, the adjusting device, in particular the adjusting member of the adjusting device, comprises a ratchet and/or toothing by means of which haptic and/or acoustic feedback is provided when generating and/or adjusting the corrective force and/or by means of which the corrective force and/or the corrected position can be fixed at an intended value.

Alternatively or additionally, the adjusting device, in particular the adjusting member of the adjusting device, can be electrically designed so that the corrective force and/or the fixed position, in particular the setting and/or generation of the corrective force and/or the corrected position, can be electrically induced and/or electrically supported. An electrical adjusting device can, for example, comprise a switch and/or a controller by means of which the generation of the corrective force and/or the movement into the corrected position can be initiated. An electrical adjusting device can, for example, comprise an electric motor and/or a rechargeable battery and/or a battery for supplying power to the adjusting device.

In a further development of the hand orthosis according to the invention, the adjusting member is designed as a rotatable adjusting member, in particular as a rotary lock. Preferably, the corrective force can be generated and/or adjusted, in particular increased and/or reduced, by a rotational movement of the adjusting member in a rotational direction. Preferably, the adjusting member is designed as a rotary knob. To fix the current corrective force and/or the currently set corrected position, the rotatable adjusting member can preferably be locked in a desired rotational position in order to block any further unintentional rotational movement of the adjusting member to change the corrective force. In addition, a locking mechanism of the adjusting member can be unlocked to unlock the adjusting member and thus to unlock the corrective force and/or the set corrected position.

For locking and/or unlocking, the adjusting member can, for example, be pulled out and/or pushed in perpendicular to the plane of rotation.

Preferably, the adjusting member has a label and/or a scale that can be used to read off a currently set corrective force value and/or the correct direction of rotation for setting the corrective force. The adjusting member can also have a textured surface, in particular a ribbed surface, which makes it easier to turn the adjusting member, for example by a patient. Alternatively, the adjusting member of the adjusting device can be designed as a linear adjusting member, in particular as a linearly adjustable adjusting member, whereby the corrective force and/or the corrected position can be adjusted by a linear movement of the adjusting member. A linearly adjustable setting element can, for example, be a setting element that is guided in a rail and can be fixed in the rail.

A hand orthosis according to the invention is also preferred, in which the adjusting device comprises a transmission member for transmitting the corrective force from the adjusting member of the adjusting device to the finger receiving element, in particular via the connecting element. Preferably, the transmission member is connected to the adjusting member of the adjusting device and/or attached to the adjusting member of the adjusting device. In particular, the transmission member can be connected to a cable of the adjusting member and/or attached to the cable of the adjusting member. If the adjusting member is designed as a rotatable adjusting member, a rotational movement of the adjusting member preferably leads to a linear movement and/or a linear extension of the transmission member, in particular to a linear force transmission, in particular a tensile force, from the adjusting member to the connecting element and/or the at least one finger receiving element of the hand orthosis. A rotation of the adjusting member thus exerts a force on the transmission member, wherein the force exerted on the transmission member can in particular correspond to the corrective force, wherein the force of the transmission member is transmitted to the connecting element and from the connecting element to the at least one finger receiving element, so that the rotational movement generated by means of the rotational movement of the adjusting member and the resulting force is transmitted via the transmission member, the connecting element and the finger receiving element to the finger and/or the hand for bringing about the corrected position.

If the adjusting member is designed as a linear adjusting member, for example as a linear fastening system, as a tensioning strap fastener and/or lashing strap fastener with ratchet, a linear movement of the adjusting member on the transmission member can be, in particular a linear movement of the transmission member and/or a force acting linearly on the transmission member, whereby the linear movement and/or the linear force of the transmission member is transmitted via the connecting element to the at least one finger receiving element.

Preferably, a tensile force is exerted on the connecting element via the movement of the transmission member. This preferably exerts a tensile force on the finger receiving element. Preferably, the transmission member and the finger receiving element and/or the transmission member and the connecting element are connected to each other and/or attached to each other. Alternatively or additionally, the adjusting device can comprise a spring, in particular a spiral spring, which generates a tensile force, in particular the corrective force, and/or realizes a force transmission to the at least one finger receiving element.

A hand orthosis according to the invention is further advantageous, in which the transmission member is formed from an elastic and/or flexible material and/or comprises an elastic and/or flexible material. Alternatively or additionally, the transmission member is formed from a non-elastic and/or non-flexible material and/or comprises a non-elastic and/or non-flexible material. Preferably, the transmission member is formed in the form of a band and/or is connected to the connecting element. In particular, the transmission member can be connected to and/or attached to the hand receiving element, which is composed of the finger receiving element and the connecting element. Preferably, the transmission member is belt-shaped, rope-shaped and/or strap-shaped. The at least one finger receiving element, the connecting element, the hand receiving element and/or the transmission member can be an integral part of a one-piece body, in particular a one-piece elastic body made of an elastic material. The transmission member is preferably designed as a rubber band. In addition, the rubber band can be made of another elastomer, of neoprene, of silicone and/or of rubber. The transmission member can be made of the same material or the same mix of materials as the connecting element and/or the at least one finger receiving element.

In a further preferred embodiment of the hand orthosis according to the invention, the hand orthosis comprises a correction indicator, via which it can be detected whether the corrective force set by means of the adjusting device and/or the resulting corrected position of the at least one finger lies within an intended correction range. Preferably, a currently set corrective force and/or a currently set corrected position can be read off via the correction indicator, for example by displaying a numerical value, for example a force in Newtons, and/or by displaying one or more signals which indicate a corrective force and/or a currently set corrected position. In particular, the correction indicator can be designed as a position indicator, which indicates whether a current finger and/or hand position corresponds to an intended finger and hand position. In addition, the correction indicator can be set up to issue a warning and/or a warning message if a current hand and/or finger position does not correspond to the intended corrected position and/or as soon as the hand and/or finger position moves out of an intended correction range. In this way, the correction indicator can be used to read and/or check whether an intended and/or correct corrective force and/or corrected position is present and/or when the intended corrected position is reached when generating and/or changing the corrective force.

Preferably, the transmission member extends through the correction indicator, whereby a movement and/or elastic elongation of the transmission member, from which the corrective force and/or the corrected position results, can lead to a change in the state of the correction indicator. By means of the correction indicator, it is preferably possible, in particular at the beginning of a therapy after the hand orthosis has been put on for the first time, to adjust the hand orthosis to the value of a corrective force suitable for the therapy and/or to bring it into an intended corrected position suitable for the therapy, in order to recognize at an early stage in the further course of the therapy as soon as a change in the corrective force and/or the corrected position leads to the finger and/or hand position threatening to move out of the intended correction range. Consequently, the correction indicator can be used to detect at an early stage when the hand position moves into the so-called drop hand position. By moving the hand and/or finger position into the drop hand position, the hand and/or fingers exert a counterforce on the adjusting device and/or the transmission member and/or the connecting element and/or the finger receiving element, whereby the counterforce can lead to the correction indicator indicating a warning that the hand is no longer in the intended position.

In another preferred embodiment of the hand orthosis according to the invention, the correction indicator is set up to generate an optical and/or haptic and/or acoustic signal relating to the set corrective force and/or the resulting corrected position, wherein the correction indicator is preferably a component of the adjusting device. In particular, the correction indicator can indicate the set corrective force and/or the resulting corrected position by means of control colors. If, for example, an intended corrective force is set and/or the intended corrected position is currently set, the correction indicator can signal that the hand orthosis, in particular the adjusting device, is correctly adjusted, for example by indicating a green control area. If the set corrective force is outside a predetermined corrective force range and/or the current corrected position is outside a predetermined position range and/or there is currently a change in the corrective force and/or the position that could cause the hand to slip into the drop hand, a red control area, for example, can indicate a need for action. In this way, it is quick and easy to read whether the hand orthosis is correctly adjusted by indicating a red or green signal from the correction indicator.

Alternatively or additionally, the correction indicator can include a red and/or green signal lamp to check the hand position. Alternatively or additionally, the correction indicator can emit a signal tone corresponding to the currently set corrective force and/or corrected position. Furthermore, when setting the corrective force and/or when setting the corrected position, haptic feedback can be emitted by the correction indicator, for example a perceptible click, which indicates that the intended corrective force and/or the intended corrected position has been reached.

In a further development of the hand orthosis according to the invention, the hand orthosis comprises a forearm receiving element which is set up to receive, in particular enclose, a forearm at least in certain areas, wherein the adjusting device is preferably arranged on the forearm receiving element and/or the forearm receiving element is connected to the finger receiving element and/or to the connecting element. The forearm receiving element of the hand orthosis is preferably designed as a forearm cuff, wherein the forearm receiving element preferably contacts, in particular encloses, the forearm at least in certain areas and/or sections. Preferably, the forearm receiving element is in close contact with the forearm at least in certain areas and/or sections. Preferably, the adjusting member and/or the correction indicator are arranged on the forearm receiving element and/or attached to the forearm receiving element. In particular, the adjusting device, especially the adjusting member and/or the correction indicator, is arranged and/or attached to the upper side of the forearm receiving element.

Preferably, the transmission member extends without contact and/or with contact to the forearm receiving element along the longitudinal extent of the forearm receiving element, in particular on the upper side of the forearm receiving element. Preferably, the forearm receiving element is fixed to the forearm in such a way that it generates a counterforce for the corrective force generated by means of the adjusting device, in particular a tensile force acting on the hand and/or the at least one finger by the adjusting device. To generate a counterforce of the forearm receiving element against the corrective force generated by the adjusting device, it should be ensured that the forearm receiving element is fixed to the forearm in such a way that it does not slip due to the effect of the corrective force on the arm.

A hand orthosis according to the invention is further preferred, in which the forearm receiving element is formed from an elastic and/or flexible material and/or comprises an elastic and/or flexible material. Alternatively or additionally, the forearm receiving element is formed from a non-elastic and/or non-flexible material and/or comprises a non-elastic and/or non-flexible material. The elastic and/or stretchable material and/or the non-elastic and/or non-stretchable material may be the same material from which the connecting element, the finger receiving element and/or the transmission member are formed. Alternatively or additionally, the forearm receiving element is formed from a breathable material and/or comprises a breathable material.

Preferably, the forearm support is made of a breathable material, in particular mesh, at least in some areas and/or at least in sections, so that sufficient ventilation of the underlying skin can be ensured despite the forearm support fitting snugly against the forearm. In particular, the forearm support can be perforated and/or include ventilation recesses, for example ventilation slits, and/or be designed with a thinner material thickness in some areas. Preferably, the forearm receiving element is designed in such a way that the forearm receiving element does not wrinkle even when force is applied, in particular when corrective force is applied, so that it is ensured that the forearm receiving element always fits snugly against the forearm and cannot slip, so that slippage of the entire hand orthosis and thus an unintentional change in the corrective force and/or the corrected position can be avoided. The forearm support can be made of mesh, felt, neoprene, leather, fabric and/or flat knit, for example, and/or comprise mesh, felt, neoprene, leather, fabric, woven fabric and/or flat knit at least in certain areas and/or sections. The forearm receiving element can be made of a single material or a mix of several different materials.

The problem underlying the invention is further solved by an orthosis assembly of the type mentioned at the beginning, wherein the stabilizing body can be reversibly detachably attached to the hand orthosis in a non-destructive manner, wherein the stabilizing body is preferably designed to immobilize the hand and/or at least one finger and/or the wrist in an intended position. Preferably, the hand orthosis of the orthosis assembly according to the invention is designed according to one of the above embodiments. With regard to the advantages and modifications of the orthosis assembly according to the invention, reference is consequently made to the advantages and modifications of the hand orthosis according to the invention.

Preferably, the stabilizing body is designed as a post-positioning splint for immobilizing the hand and/or the at least one finger, for example for night rest and/or for acute pain relief, wherein the stabilizing body can be used for immobilizing and/or fixing the hand in an intended position. Preferably, the stabilizing body is rigid, in particular in the form of a rigid splint. Preferably, the stabilizing body is replaceable and/or optionally attachable to the hand orthosis. In this way, the stabilizing body can be individually attached to and removed from the hand orthosis as required. Preferably, the hand orthosis and/or the stabilizing body comprises one or more fastening members, for example loops and/or Velcro fasteners and/or click fasteners, by means of which the stabilizing body can be reversibly and non-destructively attached to the hand orthosis. In particular, the stabilizing body can comprise one or more finger loops and/or one or more forearm loops, by means of which the stabilizing body can be attached to at least one finger and/or to the forearm by enclosing the at least one finger and/or the forearm with the respective loop in a force-fit and/or form-fit manner. The loops can, for example, be made of felt, leather and/or rubber, in particular as a felt band, leather band and/or rubber band. The loops can also be designed as a Velcro fastener and/or comprise a Velcro fastener. In addition, the stabilizing body can be designed to softly bed the at least one finger, the hand and/or the wrist as well as the forearm in an intended posture.

In a preferred embodiment of the orthosis assembly according to the invention, the stabilizing body is formed from a moldable and/or non-moldable material and/or comprises a moldable and/or non-moldable material. Alternatively or additionally, the stabilizing body contacts at least one finger and/or a palm and/or a wrist and/or a forearm at least in certain areas. Alternatively or additionally, the stabilizing body is ergonomically adapted to the at least one finger and/or the palm and/or the wrist and/or the forearm, at least in certain areas. Preferably, the stabilizing body is designed such that the at least one finger and/or the hand can be brought into an intended position and/or can be fixed in the intended position. Preferably, the stabilizing body is shaped such that the shape of the stabilizing body corresponds to a desired position of the hand and/or the at least one finger, so that the hand and/or the finger is brought into the desired position and/or fixed in the desired position by attaching the stabilizing body to the hand orthosis.

In particular, the stabilizing body is ergonomically adapted to the shape of the finger and/or the hand and/or the forearm, in particular to a neutral position. Preferably, the stabilizing body is arranged on the underside of the forearm and/or on the palm of the hand and/or on the underside of the at least one finger and/or of the at least one wrist. Preferably, the stabilizing body comprises an elastically and/or plastically formable material, in particular memory foam, sponge rubber and/or thermoplastics, at least in sections and/or regions. By using malleable materials, the stabilizing body is preferably individually adaptable to the shape of a respective finger, a respective hand and/or a respective forearm, in particular to a desired position and/or posture, and thus individually adaptable to the therapeutic needs of a respective patient. In this way, soft bedding can be achieved by using flexible materials.

The problem underlying the invention is further solved by a method for adjusting of the type mentioned at the beginning, wherein the method according to the invention comprises: generating a corrective force acting on the finger receiving element for moving the at least one finger into a corrected position by means of an adjusting device of the hand orthosis. Preferably, a hand orthosis according to one of the above embodiments is adjusted for adjustment within the scope of the method according to the invention. With regard to the advantages and modifications of the method for adjusting according to the invention, reference is therefore made to the advantages and modifications of the hand orthosis according to the invention.

In the following, preferred embodiments of the invention are explained and described in more detail with reference to the accompanying drawings. Hereby show:

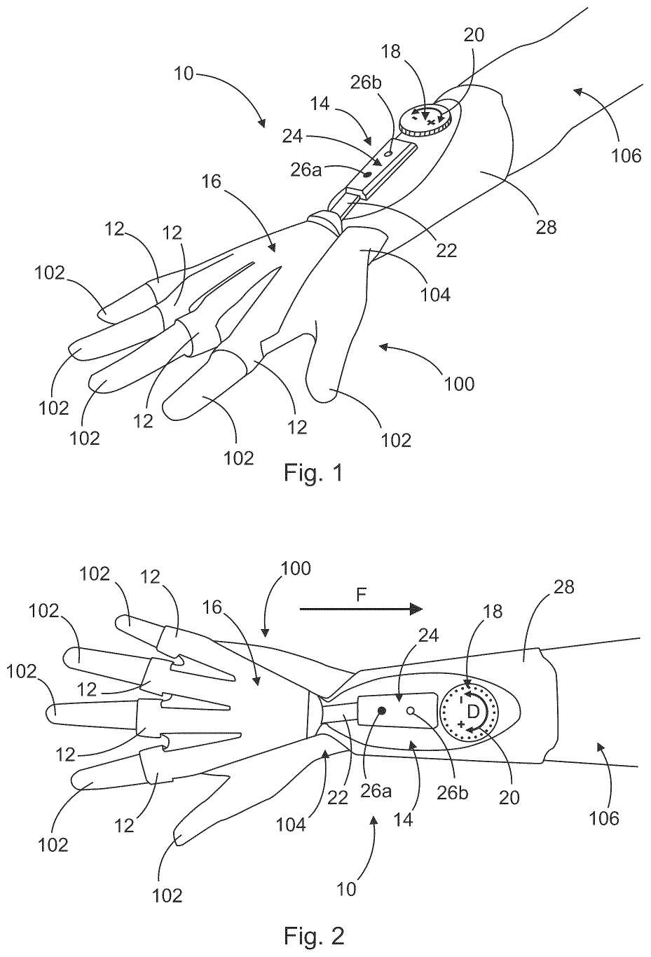

FIG. 1 a hand orthosis according to the invention in a perspective view from above;

FIG. 2 top view of the hand orthosis from FIG. 1;

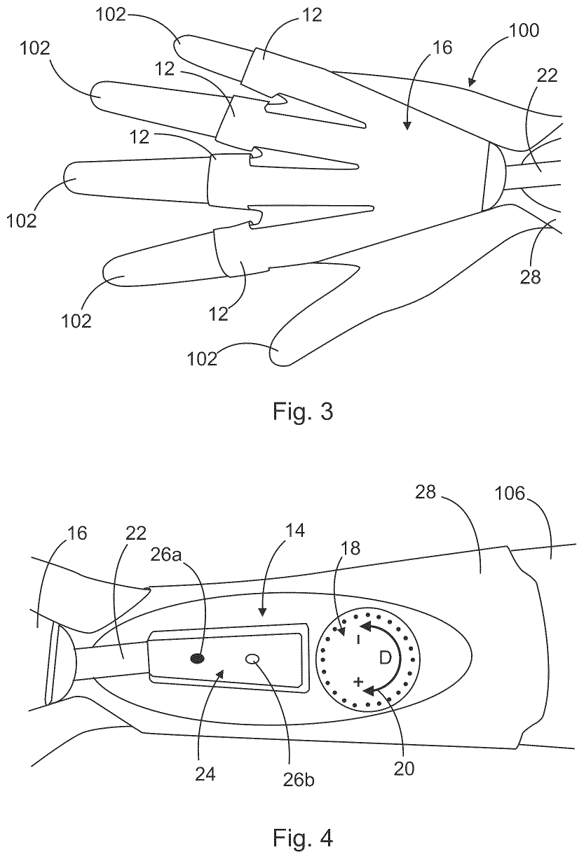

FIG. 3 finger receiving elements and a connecting element of the hand orthosis in a detailed view from above;

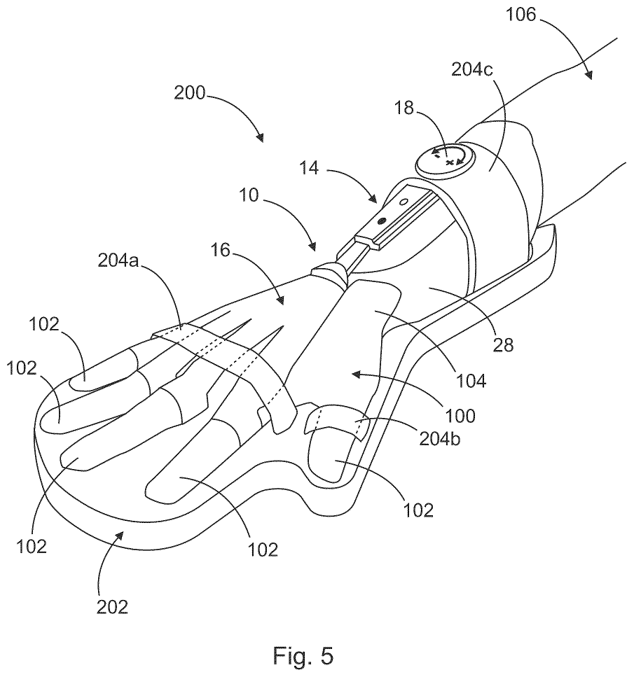

FIG. 4 a detailed view from above of an adjusting device and a forearm receiving element of the hand orthosis; and

FIG. 5 a perspective view from above of an orthosis assembly according to the invention with a hand orthosis and a stabilizing body.

FIGS. 1 and 2 show a hand orthosis 10 according to the invention, which is applied to a hand 100 and to a forearm 106, in particular of a patient. FIG. 1 shows the applied hand orthosis 10 in a perspective view and FIG. 2 shows the applied hand orthosis 10 in a top view.

The hand orthosis 10 comprises finger receiving elements 12, which receive fingers 102 of the patient's hand 100, wherein the finger receiving elements 12 are ring-shaped or loop-shaped and each finger receiving element 12 encloses and closely contacts a finger 102 in a region along the circumference of the respective finger 102. The illustrated hand orthosis 10 comprises four finger receiving elements 12, so that four fingers 102 of the hand 100 are each accommodated by one finger receiving element 12. In the illustrated embodiment of the hand orthosis 10, the little finger, the ring finger, the middle finger and the index finger are thus each enclosed by a finger receiving element 12. However, it may also be provided in other embodiments of the hand orthosis 10 that the thumb is also enclosed by a finger receiving element 12 and/or that one or more of the remaining fingers 102 are not enclosed by a finger receiving element 12.

The finger receiving elements 12 are preferably formed from an elastic and/or stretchable material, for example from an elastomer, in particular rubber, neoprene or silicone. The finger receiving elements 12 are designed to transmit a corrective force, in particular a tensile force, to the respective enclosed finger 102 in order to bring the respective finger 102 into an intended corrected position. In addition, the hand orthosis 10 comprises a connecting element 16. The connecting element 16 is preferably set up to transmit a corrective force for moving the fingers 102 by means of the finger receiving elements 12 to the finger receiving elements 12, in particular by transmitting a tensile force to the finger receiving elements 12. The connecting element 16 is formed in sections in the shape of a belt and/or band. At a first end of the connecting element 16, the connecting element 16 is connected to an adjusting device 14 of the hand orthosis 10, in particular to a transmission member 22 of the adjusting device 14, and/or to a forearm receiving element 28 of the hand orthosis 10. From this first end of the connecting element 16, the connecting element 16 extends along the back of the hand of the hand 100, wherein the connecting element contacts the back of the hand in the region of the back of the hand of the hand 100 and lies closely against the back of the hand.

Along the extension of the connecting element 16 between the first end and the finger receiving elements 12, the connecting element 16 has slit-shaped recesses in sections, which divide the connecting element 16 in regions into band-shaped sections each assigned to a finger receiving element 12, each of these sections being connected to a respective finger receiving element 12 and/or being attached to a respective finger receiving element 12. The sections of the connecting element 16 each extend in alignment and/or parallel to the direction of extension of the respective finger 102, wherein the sections of the connecting element 16 each contact and/or fit snugly against the back of the hand 100 and/or the upper side of the respective finger 102.

The connecting element 16 is formed in particular from an elastic and/or stretchable material, in particular from an elastomer such as rubber, neoprene or silicone. Preferably, the connecting element 16 is made of the same material as the finger receiving elements 12, wherein the connecting element 16 and the finger receiving elements 12 together form part of an integral body. In other embodiments of the hand orthosis 10, the connecting element 16 and the finger receiving elements 12 may also be formed as separate bodies and/or comprise different materials and/or material compositions.

The connecting element 16 is designed to transmit a corrective force generated by means of an adjusting device 14, by means of which the hand 100, a wrist 104 of the hand 100, at least one of the fingers 102 and/or the forearm 106 can be brought into a corrected position, from the adjusting device 14 to the finger receiving elements 12. The corrective force is in particular a tensile force acting in a corrective force direction, wherein the tensile force is transmitted by means of the connecting element 16 to the finger receiving elements 12 for pulling the fingers 102 into a corrected position. The adjusting device 14 is arranged and/or fastened on the forearm receiving element 28 of the hand orthosis 10, wherein the forearm receiving element 28 receives the forearm 106 along its circumference in certain regions, in particular encloses it and makes close contact over its entire surface. The forearm receiving element extends essentially from the wrist 104 along a section of the forearm 106 in the direction of the elbow.

The forearm receiving element 28 is made of a breathable material, in particular of a mesh material, at least in certain areas and/or in certain sections. In addition, the forearm receiving element 28 can be made of an elastic and/or stretchable material at least in certain areas and/or in certain sections and/or of a non-elastic or non-stretchable material at least in certain areas or in certain sections. For example, the forearm receiving element 28 can be made of mesh, felt, neoprene, leather, fabric, woven fabric and/or flat knit, at least in some areas and/or sections. Preferably, the forearm receiving element 28 is formed and/or arranged on the forearm 106 in such a way that it serves as a counterforce for the corrective force generated by the adjusting device 14, so that the generated corrective force can be transmitted to the finger receiving elements 12 with as little loss as possible, wherein the forearm receiving element 28 forms as few creases as possible under the action of force and/or does not slip substantially along the forearm 106.

The adjusting device 14 of the hand orthosis 10, by means of which the corrective force can be generated, comprises an adjusting member 18, a transmission member 22 and a correction indicator 24. The adjusting member 18 of the adjusting device 14 is designed as a rotatable adjusting member 18, in particular as a rotary fastener and/or rotary ratchet fastener, particularly preferably as a rotatable quick fastener. By rotating the rotatable adjusting member 18, the corrective force for moving the hand 100 and/or at least one finger 102 can be individually adjusted.

By turning the adjusting member 18 in the direction of rotation D, the corrective force can be increased in particular by turning in a first direction and reduced in an opposite second direction. By turning the adjusting member 18, the rotary movement of the adjusting member 18 is converted into a linear movement and/or linear extension of a transmission member 22, in particular in the direction of the corrective force, of the adjusting device 14.

The transmission member 22 is in the form of a band, in particular a rubber band, which extends from the adjusting member 18 to the first end of the connecting element 16. In particular, the adjusting member 22 extends along the longitudinal direction of the forearm 106 and/or the forearm receiving element 28 and connects the adjusting member 18 to the connecting element 16 and thus to the finger receiving elements 12. By rotating the adjusting member 18, a tensile force is generated on the transmission member 22, which is transmitted by the transmission member 22 from the adjusting member 18 to the connecting element 16 and thus to the finger receiving elements 12. In this way, a rotational movement of the adjusting member 18 leads to an increase and/or a reduction of the tensile force on the finger receiving elements 12 and thus on the fingers 102.

The pulling force can bring the fingers 102 and thus the hand 100 into an intended corrected position, so that the pulling force corresponds to a corrective force for correcting the hand and/or finger position. Thus, the hand and/or finger position can be individually adjusted by turning the adjusting member 18. By means of this individually adjustable and variable corrective force, the hand orthosis 10 is thus set up to adjust the hand 100 and/or the fingers 102 of a patient individually to a form of therapy and/or to an illness and/or to an injury in such a way that unfavorable hand positions, in particular a drop hand, are avoided and thus the success of the therapy is increased and/or a duration of therapy is shortened.

The adjusting device 14 further comprises a correction indicator 24, wherein the transmission member 22 extends through the correction indicator 24. The correction indicator 24 comprises signal indicators 26a, 26b, which indicate to a user of the hand orthosis 10, in particular to the patient adjusting the hand orthosis 10, the correct adjustment of the hand orthosis 10, in particular an intended corrective force and/or the presence of an intended corrected position of the hand 100 and/or the fingers 102. Alternatively or additionally, the signal indicators 26a, 26b indicate as soon as the corrective force moves out of a desired corrective force range and/or as soon as the hand 100 and/or the fingers 102 threaten to move out of an intended corrective position. In particular, the signal indicators 26a, 26b can be used to read whether the hand 100 is in a drop hand position that is unfavorable for therapy and/or threatens to move into a drop hand position.

In the illustrated embodiment of the hand orthosis 10, the signal indicators 26a, 26b are designed as colored signal indicators, in particular as round colored signal areas, wherein the signal indicator 26a indicates by a red signal color that the set corrective force and/or the resulting corrected position are currently in an intolerable range, and wherein the signal indicator 26b indicates by a green signal color that the set corrective force and the resulting corrected position are in a desired range. The correction indicator 24 can thus facilitate the correct setting of the hand orthosis 10 and quickly and easily recognize as soon as the setting of the hand orthosis 10 is incorrect, so that the setting of the hand orthosis 10 can be quickly corrected in such a case.

The indicated green or red signal color via the signal indicators 26a, 26b is particularly influenced by the corrective force transmitted by the transmission member 22, in particular the transmitted tensile force, since the transmission member 22 extends through the correction indicator 24 and the corrective force and/or tensile force acting on the transmission member 22 can be deduced whether the corrective force acting on the fingers 102 and/or hand 100 is correct.

The transmission member 22, which connects the adjusting member 18 to the connecting member 16, may be formed of the same material as the connecting member 16 and/or the finger receiving elements 12. The transmission member 22, the connecting member 16 and/or the finger receiving elements 12 may form a common integral body. The transmission member 22 may furthermore be formed as a separate body, which is formed from an elastic and/or stretchable material and/or from a non-elastic and/or non-stretchable material.

FIG. 3 shows a detailed view of the finger receiving elements 12 and the connecting element 16 of the hand orthosis 10 in a top view. The connecting element 16 is set up to transmit the corrective force, in particular the tensile force, which is transmitted by the transmission member 22 from the adjusting device 14, along the back of the hand of the hand 100 to the finger receiving elements 12, which each enclose a finger 102. The connecting element 16 is connected at its right first end to the transmission member 22 and/or is attached to the transmission member 22. Along the back of the hand 100, the connecting element 16 is slotted at the region of the connecting element 16 facing the fingers 102 in its longitudinal extension, in particular in the parallel direction to the longitudinal extension of the fingers 102, such that four band-shaped regions of the connecting element 16, which each extend in the direction of one of the fingers 102 of the hand 100, are formed, wherein a finger receiving element 12 for enclosing a respective finger 102 is arranged at the end of each band-shaped section of the connecting element 16.

In the embodiment shown, the connecting element 16 and the finger receiving elements 12 form a one-piece integral body and are formed from the same elastic and extensible material. In this way, a corrective force is transmitted from the adjusting device 14 via the transmission member 22 to the connecting element 16 and is in turn transmitted from the connecting element 16 to the finger receiving elements 12, so that a corrective force generated by the adjusting device 14, in particular a tensile force, acts in the corrective force direction F on the fingers 102 of the hand 100 in such a way that the fingers 102 and thus the entire hand 100 can be brought into an intended corrected position by the corrective force.

FIG. 4 shows a detailed view of the forearm receiving element 28 and the adjusting device 14 of the hand orthosis 10 arranged thereon in a top view. The adjusting device 14 is arranged on the upper side of the forearm receiving element 28, which contacts the upper region of the forearm 106. The adjusting member 20, which is designed as a rotatable adjusting member 20, can be rotated clockwise and/or counterclockwise in the direction of rotation D. The adjusting member 18 comprises a label 20 which indicates the direction of rotation D and indicates by means of a plus sign and a minus sign in which direction of rotation D the corrective force generated by the adjusting member 18, in particular the tensile force exerted on the transmission member 22, is increased or reduced. The adjusting member 18 also comprises a corrugation along its outer circumference, which in the embodiment shown also has a scale with graduation marks, which makes it easier to turn the adjusting member 18.

If the adjusting member 18 is turned clockwise in the direction of rotation D, the rotary movement of the adjusting member 18 is converted into a linear movement of the transmission member 22, with the adjusting member 18 pulling on the adjusting member 22 as a result of the rotary movement. The tensile force on the transmission member 22 pulls on the connecting element 16 connected to the transmission member 22. In this way, the corrective force generated by the adjusting device 14 is transmitted to the finger receiving elements 12 of the hand orthosis 10 in the form of a tensile force via the transmission member 22 and the connecting element 16.

The band-shaped transmission member 22, which is formed from an elastic material, extends along the longitudinal extent of the forearm receiving element 28 from the adjusting member 18 to the connecting element 16, wherein the transmission member 22 extends along its longitudinal extent through the correction indicator 24. The tensile force acting on the transmission member 22 and the resulting elongation of the transmission member 22 and/or the resulting linear movement and/or linear elongation of the transmission member 22 leads to a change in state of the correction indicator 24, in particular to a change in state and/or a signal change in the signals indicated by signal indicators 26a, 26b for detecting and/or reading a currently set corrective force, a currently present corrected position resulting from the current corrective force and/or a current change in the corrective force and/or a current change in the corrected position resulting from the change in the corrective force.

The signal indicators 26a, 26b of the correction indicator 24 indicate the current status of the hand orthosis 10 by means of a colored signal. The signal indicator 26a uses a red color to indicate when the set corrective force is outside an intended corrective force range. In addition, the indicator of a red color by the signal indicator 26a can indicate when the hand 100 and/or the fingers 102 are currently not in an intended corrected position, for example in a drop hand position. The signal indicator 26b may use a green color to indicate when the set corrective force is currently in a desired corrective force range and/or that the hand 100 and/or fingers 102 are currently set in an intended corrected position. In other embodiments of the hand orthosis 10, the indication of the corrective force and/or the intended position may also be indicated by signal indicators formed in a different manner. It is conceivable that the correction indicator 24 in other embodiments may, for example, have a loudspeaker for emitting signal tones and/or a indicator for indicating numerical values, for example for indicating a currently set corrective force, and/or signal lights which indicate the current status of the hand orthosis 10 by lighting up, in particular in different colors

The adjusting device 14 is preferably designed in such a way that a desired generated corrective force and/or a corrected position resulting from the corrective force is locked and/or fixed at a desired value. For this purpose, for example, the adjusting member 18 can be designed to remain in a current position and a resulting corrective force as long as no external force acts on the adjusting member 18, for example by manually turning the adjusting member 18. For this purpose, the adjusting member 18 can have an internal mechanism, for example a toothing, which can also be locked and/or unlocked by manually locking and unlocking the adjusting member, for example by pulling out or pushing in the adjusting member perpendicular to the direction of rotation, in order to fix and/or release a currently set corrective force and/or a currently set corrected position.

FIG. 5 shows an orthosis assembly 200 according to the invention, which comprises a hand orthosis 10, in particular a hand orthosis 10 as shown in FIGS. 1 to 4, and which comprises a stabilizing body 202. The orthosis assembly 200 with the hand orthosis 10 and the stabilizing body 202 is shown in FIG. 5 in a perspective view from above.

The stabilizing body 202 is designed as a support for the hand 100, the fingers 102 and a section of the forearm 106, in particular as a splint-shaped support, particularly preferably as a secondary support splint. The stabilizing body 202 extends from the end of the forearm receiving element 28 of the hand orthosis 10 facing the elbow to the fingertips of the fingers 102, wherein the stabilizing body 202 is arranged on the underside of the forearm 106 and the hand 100. In particular, the stabilizing body 202 contacts the inner surface of the hand 100, the inner surfaces of the fingers 102 and the inner surface of the wrist 104 and the forearm 106 at least in certain areas. The hand 100 as well as the forearm 106, the fingers 102 and the wrist 104 lie flat on the stabilizing body 202.

The stabilizing body 202 is attached to the hand orthosis 10 and/or to the hand 100 and/or to the forearm 106 via fastening members 204a-204c. The fastening members 204a-204c are loop-shaped. The fastening member 204a extends in the transverse direction to the longitudinal extension of the hand 100 over the back of the hand 100 and over the connecting element 16, in particular over the band-shaped sections of the connecting element 16, and contacts the connecting element 16 and/or the back of the hand 100 at least in certain areas. In particular, a compressive force is exerted from above on the connecting element 16 and on the hand 100 via the fastening member 204a, so that the palm of the hand 100 is at least slightly pressed against the stabilizing body 202. The fastening member 204b extends in the transverse direction to the longitudinal extension of the thumb of the hand 100, so that the thumb is also pressed onto the stabilizing body 202 with at least a slight compressive force. The fastening member 204c preferably extends in the transverse direction to the longitudinal extension of the forearm 106 and in particular contacts the forearm receiving element 28 at least in some areas and the adjusting device 14 at least in some areas. The fastening member 204c is preferably wider than the fastening members 204a and 204b, wherein the fastening member 204 extends at least in sections in the circumferential direction around the lower arm receiving element 28 and exerts at least a slight compressive force on the lower arm in the direction of the stabilizing body 202, so that the lower arm 106 is pressed with its underside against the stabilizing body 202.

The fastening members 204a-204c are each formed as band-shaped loops, wherein the fastening members 204a-204c may be formed from a stretchable and/or elastic and/or from a non-stretchable and/or non-elastic material. The fastening members 204a-204c comprise, for example, felt, leather and/or rubber. Preferably, the fastening members 204a-204c are formed as a felt band, leather band and/or rubber band, wherein the material is formed in particular to be breathable, for example in that slit-shaped and/or circular recesses may be arranged in the fastening members 204a-204c, through which the fastening members 204a-204c become permeable to air. The fastening members 204a-204c can also comprise a mesh material and/or comprise mesh material at least in sections.

The stabilizing body 202 is thus reversibly non-destructively detachably connected to the hand orthosis 10 via the fastening members 204a-204c, so that the stabilizing body 202 is configured as a removable and/or replaceable post-support splint. The stabilizing body 202 is particularly adapted to immobilize and/or stabilize the fingers 102, the hand 100, the wrist 104 and/or the forearm 106, particularly in combination with the hand orthosis 10 to support the hand orthosis 10, in an intended posture. For example, the stabilizing body 202 can be used to relieve pain and/or provide soft bedding at the beginning of a therapy by providing additional immobilization.

The stabilizing body 202 is formed at least in some areas from malleable and/or yielding materials, for example memory foam, foam rubber and/or thermoplastics, so that the stabilizing body 202 is ergonomically adaptable to the hand 100, the fingers 102 and/or the forearm 106 and/or automatically adapts to the contours of the hand 100, the fingers 102 and/or the forearm 106 when the stabilizing body 202 is put on by deformation of the material of the stabilizing body 202. The stabilizing body 202 can thus conform to the contours. In this way, the hand 100 with the fingers 102 and/or with the forearm 106 can be brought into a desired position, in particular into a neutral position, in order to support the therapy by means of the hand orthosis 10.

### REFERENCE SIGN

10 Hand orthosis
12 Finger receiving element
14 Adjusting device
16 Connecting element
18 Adjusting member
20 Labeling
22 Transmission member
24 Correction indicator
26a, 26b Signal indicator
28 Forearm receiving element
100 Hand
102 Fingers
104 Wrist
106 Forearm
200 Orthosis assembly
202 Stabilizing body
204a-204c Fastening member
D Direction of rotation
F Corrective force direction

1. A hand orthosis comprising:
at least one finger receiving element, configured to receive, at least one finger of a hand at least in certain areas;
wherein an adjusting device is configured to generate a corrective force acting on the finger receiving element, thereby the at least one finger is brought into a corrected position.

2. The hand orthosis according to claim 1, wherein at least one connecting element connects the adjusting device to the at least one finger receiving element and is configured to transmit the corrective force generated by the adjusting device to the finger receiving element.

3. The hand orthosis according to claim 2, wherein the connecting element
is formed from an elastic or flexible material or comprises an elastic or flexible material, or
is formed from a non-elastic or non-flexible material or comprises a non-elastic or non-flexible material.

4. The hand orthosis according to claim 1,
wherein the adjusting device is configured as a mechanical adjusting device, wherein
the corrective force is generated or adjusted by an adjusting member of the adjusting device, or
the adjusting member of the adjusting device is fixed in a desired position for fixing an intended corrective force.

5. The hand orthosis according to claim 4, wherein the adjusting member is a rotatable adjusting member.

6. The hand orthosis according to claim 4,
wherein the adjusting device comprises a transmission member for transmitting the corrective force from the adjusting member of the adjusting device to the finger receiving element.

7. The hand orthosis according to claim 6, wherein the transmission member
is formed from an elastic or flexible material or comprises an elastic or flexible material, or
is formed from a non-elastic or non-flexible material or comprises a non-elastic or non-flexible material.

8. The hand orthosis according to claim 1,
wherein a correction indicator is the corrective force set by the adjusting device, or the resulting corrected position of the at least one finger lies within an intended correction range.

9. The hand orthosis according to claim 8, wherein the correction indicator is set up to generate at least one of an optical, a haptic, and an acoustic signal relating to the set corrective force or the resulting corrected position.

10. The hand orthosis according to claim 1,
wherein a forearm receiving element is configured to receive a forearm at least in certain areas.

11. The hand orthosis according to claim 10, wherein the forearm receiving element
is formed from an elastic or flexible material or comprises an elastic or flexible material, or
is formed from a non-elastic or non-flexible material or comprises a non-elastic or non-flexible material, or
is formed from a breathable material or comprises a breathable material.

12. An orthosis assembly comprising:
a hand orthosis, comprising:
at least one finger receiving element, configured to receive at least one finger of a hand at least in certain areas, wherein an adjusting device is configured to generate a corrective force acting on the finger receiving element thereby the at least one finger is brought into a corrected position; and
a stabilizing body arranged on the hand orthosis,
wherein the stabilizing body is reversibly detachably attached to the hand orthosis in a non-destructive manner.

13. The orthosis assembly according to claim 12,
wherein the stabilizing body is configured to or configured to be at least one of:
formed from a moldable or non-moldable material or comprises a moldable or non-moldable material,
contact at least one finger, and/or a palm, a wrist, and a forearm at least in certain areas, and
ergonomically adapted, at least in certain areas, to the at least one finger, the palm, the wrist, and the forearm.

14. A method for adjusting a hand orthosis, in comprising:
providing, at least one finger receiving element, configured to receive at least one finger of a hand at least in certain areas, wherein an adjusting device is configured to generate a corrective force acting on the finger receiving element thereby the at least one finger is brought into a corrected position;
receiving at least in certain areas, at least one finger of a hand by the at least one finger receiving element of the hand orthosis,
wherein:
generating a corrective force acting on the finger receiving element for moving the at least one finger into a corrected position by an adjusting device of the hand orthosis.
